(19) United States
(12) Patent Application Publication
(10) Pub. No.: US 2024/0269053 A1
(43) Pub. Date: Aug. 15, 2024

(54) COMPOSITION FOR CLEANSING AND/OR REMOVING MAKEUPS FROM KERATIN MATERIALS

(71) Applicant: L'OREAL, Paris (FR)

(72) Inventor: Lingwei XU, Shanghai (CN)

(73) Assignee: L'OREAL, Paris (FR)

(21) Appl. No.: 18/561,085

(22) PCT Filed: Jun. 24, 2021
(86) PCT No.: PCT/CN2021/102116

§ 371 Date: Nov. 15, 2023

(51) Int. Cl. A61K 8/44; A61K 8/34; A61K 8/46; A61K 8/60; A61Q 19/10

(52) U.S. Cl. CPC A61K 8/442; A61K 8/345; A61K 8/463; A61K 8/60; A61Q 19/10

(57) ABSTRACT

A composition for cleansing and/or removing makeups from keratin materials, comprises in an aqueous phase: a) at least one amino acid surfactant; b) at least one chelating agent selected from aminocarboxylic acids and salts thereof; and c) at least one divalent metal salt. A non-therapeutic process for cleansing and/or removing makeups from keratin materials, in particular the skin, comprises the application to the keratin materials, in particular the skin, of the composition, and rinsing off said composition after an optional period of time.

### TECHNICAL FIELD

The present invention relates to a cosmetic composition, in particular, a composition for cleansing and/or removing makeups from keratin materials. The present invention also relates to a non-therapeutic process for cleansing and/or removing makeups from keratin materials using said composition.

### BACKGROUND ART

Cleansing the skin or removing makeups from the skin is very important for caring for the skin. It must be as efficient as possible because greasy residues, such as excess sebum, the remnants of cosmetic products used daily and make-up products, in particular waterproof products, accumulate in the skin folds, and can block the pores of the skin and result in the appearance of spots.

Several types of skin cleansing or makeup removing products, for example, rinsable anhydrous oils and gels, foaming creams and lotions, are known.

Rinsable anhydrous oils and gels have a cleansing or makeup removing action by virtue of oils present in these formulations. These oils make it possible to dissolve fatty residues and to disperse make-up pigments. These products are effective and well tolerated.

However, they exhibit the disadvantages of being heavy, of not foaming and of not conferring a good skin finish upon application, which is disadvantageous from a cosmetic viewpoint.

On the other hand, foaming creams, lotions and gels have a cleansing or makeup removing action by virtue of the surfactants, which suspend the fatty residues on the face. They are effective and pleasant to use because the foam and they are easy to remove.

Foam property is of great importance for the foaming cleanser. However, some foaming cleansers are not satisfying in terms of foam density.

In order to have better foam density in a foaming cleanser containing amino acid surfactant, traditional methods may include more surfactants or more polymers. However, these methods would increase the cost of formulation or bring undesired rinse-off sensory and skin finish.

Therefore, there is a need to provide another solution on improving foam density.

### SUMMARY OF THE INVENTION

The inventors have found that such a need can be achieved by the present invention.

Thus, according to an aspect, the present invention provides a composition for cleansing and/or removing makeups from keratin materials, comprising in an aqueous phase:
a) at least one amino acid surfactant;
b) at least one chelating agent selected from aminocarboxylic acids and salts thereof; and
c) at least one divalent metal salt.

The composition according to the present invention can provide a good foam density.

It may be used as a daily facial cleanser or a makeup remover. The composition of the present invention is a rinse-off product. Thus, such a composition can be applied on the skin (i.e. face and/or body), and then rinsed with flush water.

According to another aspect, the present invention provides a non-therapeutic process for cleansing and/or removing makeups from keratin materials, in particular the skin, comprising the application to the keratin materials, in particular the skin, of the composition according to the present invention, and rinsing off said composition after an optional period of time.

Other subjects and characteristics, aspects and advantages of the invention will emerge even more clearly on reading the description and the examples that follows.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values, which are capable of being changed according to the desired purpose as required.

For the purposes of the present invention, the term “keratin material” is intended to cover human skin, mucous membranes such as the lips. Facial skin are most particularly considered according to the present invention.

All percentages in the present invention refer to weight percentage, unless otherwise specified.

According to an aspect, the present invention relates to a composition for cleansing and/or removing makeups from keratin materials, comprising in an aqueous phase:
a) at least one amino acid surfactant;
b) at least one chelating agent selected from aminocarboxylic acids and salts thereof; and
c) at least one divalent metal salt.

### Amino Acid Surfactants

The composition according to the present invention comprises at least one amino acid surfactant.

In one embodiment, said amino acid surfactant is derived from a carboxylate salt of amino acid wherein the amino group situated on the α-carbon or β-carbon of an amino acid salt is acylated with a C₅-C₂₂ fatty acid derivative.

The carboxylate salts of these amino acids can be formed by conventional means such as by neutralization of the respective amino acid with a base. The amino group situated on the α-carbon or β-carbon of the neutralized amino acid is acylated with a fatty acid halide (acyl halide) in the presence of a base via the well-known Schotten-Baumann reaction giving the amide, thus forming the desired surfactant reaction product, i.e. the amino acid surfactant. Suitable acyl halides for acylation of the amino acid carboxylate salt include acyl chlorides, bromides, fluorides, and iodides. The acyl halides can be prepared by reacting a saturated or unsaturated, linear or branched C₈-C₂₂ fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in US Patent Application Publication No. 2008/0200704, published on Aug. 21, 2008, which application is incorporated herein by reference.

Preferably, said amino acid surfactant is represented by the formula (I):

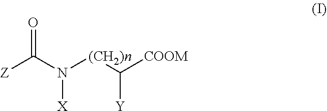

wherein:
Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)OH, —(CH₂)₂C(O)O⁻M⁺, and
M is a salt-forming cation wherein COO is the counter-anion, such as for example sodium, potassium, ammonium, or triethanolamine.

In one embodiment, in formula (I):
Z represents a linear or branched C₈-C₂₂ alkyl group,
X is a hydrogen or methyl group,
n is 0,
Y is selected from hydrogen, —(CH₂)₂C(O)OH, —(CH₂)₂C(O)O˜M⁺, and
M is a salt-forming cation wherein COO is the counter-anion, such as sodium, potassium, ammonium, or triethanolamine.

According to a preferred embodiment of the invention, in formula (I):
Z represents a linear or branched C₈-C₂₂ alkyl group,
X is a hydrogen or methyl group,
n is 0,
Y is selected from hydrogen, —(CH₂)₂C(O)OH, —(CH₂)₂C(O)O⁻M⁺, and
M is a salt-forming cation wherein COO is the counter-anion, such as for example sodium, potassium, ammonium, or triethanolamine.

Examples of the amino acid surfactants are salt of alanine, arginine, aspartic acid, glutamic acid, glycine, isoleucine, leucine, lysine, phenylalanine, serine, tyrosine, valine, sarcosine, and any mixture thereof. More specifically, mentions can be made of the amino acid surfactants such as dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

References can be made to the commercially available amino acid surfactant of, for example, acylsarcosinates, for instance the sodium lauroyl sarcosinate sold under the name Sarkosyl NL 97® by the company Ciba or sold under the name Oramix L 30® by the company SEPPIC, the sodium myristoyl sarcosinate sold under the name Nikkol Sarcosinate MN® by the company Nikkol or the sodium palmitoyl sarcosinate sold under the name Nikkol Sarcosinate PN® by the company Nikkol; alaninates, for instance the sodium N-lauroyl-N-methylamidopropionate sold under the name Sodium Nikkol Alaninate LN 30® by the company Nikkol or sold under the name Alanone ALE® by the company Kawaken, and the N-lauroyl-N-methylalanine triethanolamine sold under the name Alanone Alta® by the company Kawaken; N-acylglutamates, for instance the triethanolamine monococoylglutamate sold under the name Acylglutamate CT-12® by the company Ajinomoto and the triethanolamine lauroylglutamate sold under the name Acylglutamate LT-12® by the company Ajinomoto; glycinates, for instance sodium N-cocoylglycinate sold under the name Amilite GCS-12® by the company Ajinomoto; aspartates, for instance the mixture of triethanolamine N-lauroyl aspartate and of triethanolamine N-myristoylaspartate, sold under the name Asparack® by the company Mitsubishi; citrates, and any mixture thereof.

According to the present invention, the preferred amino acid surfactant is selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, or a mixture thereof.

Mentions of the preferred amino acid surfactant in the composition which is commercially available can be made to sodium lauroyl sarcosinate (ORAMIX L 30 sold by Seppic), sodium cocoyl glycinate (and) water (AMILITE® GCS-12K sold by Ajinomoto), sodium cocoyl glutamate (and) disodium cocoyl glutamate (AMISOFT® CS-22 sold by Ajinomoto), and sodium lauroyl glutamate (AMISOFT® LS-11 sold by Ajinomoto).

According to a preferred embodiment, the amino acid surfactant is selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof.

Due to the use of amino acid surfactant, the composition according to the present invention brings no irritation to keratin materials.

Advantageously, the amino acid surfactant is present in the composition according to the present invention in an amount ranging from 1 wt. % to 60 wt. %, preferably from 20 wt. % to 50 wt. %, more preferably from 35 wt. % to 45 wt. % relative to the total weight of the composition.

### Chelating Agent

The composition according to the present invention comprises at least one chelating agent selected from aminocarboxylic acids and salts thereof.

The salts are especially alkali metal, alkaline-earth metal, ammonium and substituted ammonium salts.

The chelating agents may be selected in particular from the compounds having the following INCI name:
diethylenetriaminepentaacetic acid (DTPA),
ethylenediaminedisuccinic acid (EDDS) and trisodium ethylenediamine disuccinate such as Octaquest E30 from INNOSPEC ACTIVE CHEMICALS,
ethylenediaminetetraacetic acid(EDTA),
ethylenediamine-N, N′-diglutaric acid (EDDG),
glycinamide-N,N′-disuccinic acid (GADS),
2-hydroxypropylenediamine-N,N′-disuccinic acid (HPDDS),
ethylenediamine-N,N′-bis(ortho-hydroxyphenylacetic acid) (EDDHA),
N,N′-bis(2-hydroxybenzyl)ethylenediamine-N,N′-diacetic acid (HBED),
nitrilotriacetic acid (NTA),
methylglycine diacetic acid (MGDA),
N-2-hydroxyethyl-N,N-diacetic acid and glyceryl imino diacetic acid (as described in documents EP-A-317 542 and EP-A-399 133),
iminodiacetic acid-N-2-hydroxypropyl sulfonic acid and aspartic acid N-carboxymethyl N-2-hydroxypropyl-3-sulfonic acid (as described in EP-A-516 102),
beta-alanine-N,N′-diacetic acid, aspartic acid-N,N′-diacetic acid and aspartic acid-N-monoacetic acid (described in EP-A-509 382),
chelating agents based on iminodisuccinic acid (IDSA) (as described in EP-A-509 382),
ethanoldiglycine acid, and
tetrasodium glutamate diacetate (GLDA) such as Dissolvine GL38 or 45S from Akzo Nobel.

Among the chelating agents mentioned, ethylenediamine-tetraacetic acid (EDTA), diethylenetriaminepentaacetic acid (DTPA), S,S′-ethylenediaminedisuccinic acid(EDDS), trisodium ethylenediamine disuccinate, ethylenediaminetetramethylenephosphonic acid (EDTMP), and tetrasodium glutamate diacetate (GLDA), and mixtures thereof, are preferably used.

Advantageously, the chelating agent is present in an amount ranging from 0.05 wt. % to 5 wt. %, preferably from 0.1 wt. % to 1 wt. %, and more preferably from 0.2 wt. % to 0.4 wt. %, relative to the total weight of the composition.

### Metal Salts

The composition according to the present invention comprises at least one divalent metal salt.

The divalent and/or trivalent metal salt comprises a metal ion M1²⁺. Preferably, M1²⁺ is selected from Mg²⁺, Ca²⁺, Zn²⁺, Fe²⁺, Mn²⁺, and Cu^{2′}.

The metal salt is selected from metal salts of an organic acid or an inorganic acid.

As example of organic acid, mention can be made of ascorbic acid, formic acid, acetic acid, glycolic acid, gluconic acid, lactic acid, mandelic acid, oxalic acid, maleic acid, malonic acid, glyoxylic acid, succinic acid, adipic acid, fumaric acid, sebacic acid, citric acid, tartaric acid, malic acid, tricarboxylic acid, glutaric acid, glucaric acid, pyrrolidone carboxylic acid, phenol sulfonic acid, salicylic acid, etc.

As example of inorganic acid, mention can be made of sulfuric acid, carbonic acid, silicic acid, hydrochloric acid, nitric acid, phosphoric acid, etc.

Preferably, metal salts of an inorganic acid are selected from metal chlorides, sulfates, nitrates, carbonates and hydrogen carbonates, phosphates, silicates, and mixtures thereof, wherein the metal is selected from Mg, Ca, Zn, Mn, Cu, and Fe.

More preferably, metal salts of an inorganic acid are selected from calcium chloride, calcium sulfate, calcium nitrate, calcium carbonate and hydrogen carbonate, calcium phosphate, zinc chloride, zinc sulfate, zinc nitrate, zinc carbonate and hydrogen carbonate, zinc phosphate, magnesium chloride, magnesium sulfate, magnesium nitrate, magnesium carbonate and hydrogen carbonate, magnesium phosphate, magnesium aluminum silicate, and mixtures thereof.

Preferably, metal salts of an organic acid are selected from metal ascorbates, formates, acetates, glycolates, gluconates, lactates, mandelates, oxalates, maleates, malonates, glyoxylates, succinates, adipates, fumarates, sebacates, citrates, tartarates, malates, tricarboxylates, glutarates, glucarates, pyrrolidone carboxylates, phenolsulfonate, salicylates, and mixtures thereof, wherein the metal is selected from Mg, Ca, Zn, Mn, Cu, and Fe.

More preferably, metal salts of an organic acid are selected from magnesium gluconate, magnesium PCA (Magnesium pyrrolidone carboxylate), magnesium acetate, calcium PCA(calcium pyrrolidone carboxylate), zinc lactate, zinc gluconate, zinc phenolsulfonate, zinc salicylate, Zinc PCA (zinc pyrrolidone carboxylate), zinc citrate, zinc ascorbate, copper PCA (copper pyrrolidone carboxylate), copper gluconate, and mixtures thereof.

According to a preferred embodiment, the metal salt is selected from magnesium gluconate, magnesium PCA, magnesium sulfate, magnesium acetate, magnesium carbonate, magnesium aluminum silicate, calcium carbonate, calcium PCA, calcium chloride, calcium carbonate, zinc PCA, zinc gluconate, copper PCA, copper gluconate, and a mixture thereof.

Advantageously, the metal salt is present in the composition according to the present invention in an amount ranging from 0.1 wt. % to 5 wt. %, preferably from 0.3 wt. % to 3 wt. %, more preferably from 0.5 wt. % to 1 wt. %, relative to the total weight of the composition.

### Aaueous Phase

The composition of the present invention comprises at least one aqueous phase.

According to a preferred embodiment, the composition of the present invention is an aqueous paste.

The aqueous phase of the composition according to the present invention comprises water and optionally one or more water-miscible or at least partially water-miscible compounds, for instance C2-C8 polyols or monoalcohols, such as ethanol and isopropanol.

The term “polyol” should be understood as meaning any organic molecule comprising at least two free hydroxyl groups. Examples of polyols that may be mentioned include glycols, for instance butylene glycol, propylene glycol, dipropylene glycol, isoprene glycol, hexylene glycol, caprylyl glycol, glycerol (i.e. glycerin) and polyethylene glycols.

According to a preferred embodiment, the composition according to the present application further comprises at least one C2-C8 polyol selected from butylene glycol, propylene glycol, dipropylene glcol, isoprene glycol, hexylene glycol, caprylyl glycol, glycerin and polyethylene glycols. More preferably, the C2-C8 polyol is glycerin.

Preferably, the C2-C8 polyol is present in an amount ranging from 1 wt. % to 70 wt. %, preferably from 30 wt. % to 40 wt %, relative to the total weight of the composition.

The aqueous phase may represent from 40 wt. % to 70 wt. %, preferably from 45 wt. % to 65 wt. %, relative to the total weight of the composition.

### Additional Ingredients

The composition according to the present invention may comprise one or more additional ingredients, selected from those conventionally used in skin cleanser.

The composition in accordance with the present invention may comprise any of the following additives: pH adjusting agents (e.g. citric acid); additional surfactants; biological extracts; antibacterial agents, fragrances; thickeners (such as acrylates copolymer); structuring agents (such as potassium chloride, cetyl alcohol), and cationic preservatives.

A person skilled in the art can adjust the type and amount of additional ingredients present in the compositions according to the present invention by means of routine operations, so that the desired properties of these compositions are not adversely affected by the additional ingredients.

According to a preferred embodiment, the present invention relates to a composition for cleansing and/or removing makeups from keratin materials, comprising in an aqueous phase, relative to the total weight of the composition:
a) from 35 wt. % to 45 wt. % of at least one amino acid surfactant selected from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof;
b) from 0.2 wt. % to 0.4 wt. % of at least one chelating agent selected fromethylenediamine-tetraacetic acid (EDTA), diethylenetriaminepentaacetic acid (DTPA), S,S′-ethylenediaminedisuccinic acid(EDDS), trisodium ethylenediamine disuccinate, ethylenediaminetetramethylenephosphonic acid (EDTMP), and tetrasodium glutamate diacetate (GLDA), and mixtures thereof; and
c) from 0.5 wt. % to 1 wt. % of at least one divalent metal salt selected from magnesium gluconate, magnesium PCA, magnesium sulfate, magnesium acetate, magnesium carbonate, magnesium aluminum silicate, calcium carbonate, calcium PCA, calcium chloride, calcium carbonate, zinc PCA, zinc gluconate, copper PCA, copper gluconate, and a mixture thereof.

The composition according to the present invention may comprises an oil. Preferably, the composition according to the present invention does not comprise any oil.

### Process and Use

The composition according to the present invention can be used in a process for cleansing and/or removing makeups from keratin materials, such as the skin, in particular the face, by being applied to the keratin materials.

The composition according to the present invention can be a crystalized system in the form of a paste, a cream or a gel textured lotion.

It was found that the composition according to the present invention can provides a good foam density.

The composition according to the invention may be applied by any means enabling a uniform distribution, in particular using a finger, or a cotton ball, and can be removed by rinsing with water.

Thus, according to another aspect, the present invention relates to a non-therapeutic process for cleansing and/or removing makeups from keratin materials, in particular the skin, comprising the application to the keratin materials, in particular the skin, of the composition according to the present invention, and rinsing off said composition after an optional period of time.

The present invention is illustrated in greater detail by the examples described below, which are given as non-limiting illustrations.

### EXAMPLES

Main raw materials used, trade names and supplier thereof are listed in Table 1.

TABLE 1

| INCI Name | Trade Name | Supplier |
|---|---|---|
| SODIUM COCOYL | AMILITE ® GCS-12K | AJINOMOTO |
| GLYCINATE | | |
| GLYCERIN | GLICENAT ® GC K MB | OXITENO |
| POTASSIUM | CLORETO DE POTASSIO | LABSYNTH |
| CHLORIDE | U.S.P/F.C.C | |
| CETYL ALCOHOL | LANETTE 16 | BASF |
| CITRIC ACID | CITRIC ACID MONOHYDRATE | CITRIQUE |
| | GRANULAR | BELGE |
| ACRYLATES | CARBOPOL ® AQUA SF-1 | LUBRIZOL |
| COPOLYMER | POLYMER | |
| TRISODIUM | NATRLQUEST ® E30 | INNOSPEC |
| ETHYLENE- | | ACTIVE |
| DIAMINE | | CHEMICALS |
| DISUCCINATE | | |
| MAGNESIUM | GLUCONAL ® MG-P | PURAC |
| GLUCONATE | | |
| COPPER | MINERALIS GU/CU | GIVAUDAN- |
| GLUCONATE | | LAVIROTTE |
| SODIUM | API-NACL PHARMACOPOEIA | ESCO |
| CHLORIDE | SODIUM CHLORIDE GMP | |
| | GRADE | |
| MAGNESIUM | RONACARE ® MAGNESIUM | MERCK |
| SULFATE | SULFATE | |

### Invention Examples 1-4 and Comparative Example 1-4

The composition of invention examples (IE) 1-4 and comparative examples (CE) 1-4 and comprising the ingredients shown in Table 2 were prepared, with all amounts expressed by percentages by weight of active matter with regard to the total weight of each composition.

TABLE 2

| Components | IE. 1 | IE. 2 | IE. 3 | IE. 4 | CE. 1 | CE. 2 | CE. 3 | CE. 4 |
|---|---|---|---|---|---|---|---|---|
| SODIUM COCOYL | 40 | 40 | 40 | 40 | 40 | 40 | 40 | 40 |
| GLYCINATE | | | | | | | | |
| GLYCERIN | 35 | 35 | 35 | 35 | 35 | 35 | 35 | 35 |
| WATER | QS 100 | QS 100 | QS 100 | QS 100 | QS 100 | QS 100 | QS 100 | QS 100 |
| TRISODIUM | 0.2 | 0.2 | 0.2 | 0.4 | 0.2 | | 0.2 | |
| ETHYLENEDIAMINE | | | | | | | | |
| DISUCCINATE | | | | | | | | |
| MAGNESIUM | 0.5 | | | 0.5 | | 0.5 | | |
| GLUCONATE | | | | | | | | |
| COPPER | | 0.5 | | | | | | |
| GLUCONATE | | | | | | | | |
| MAGNESIUM | | | 0.5 | | | | | |
| SULFATE | | | | | | | | |
| SODIUM CHLORIDE | | | | | | | 0.5 | |
| ACRYLATES | 5 | 5 | 5 | 5 | 5 | 5 | 5 | 5 |
| COPOLYMER | | | | | | | | |
| POTASSIUM | 2 | 2 | 2 | 2 | 2 | 2 | 2 | 2 |
| CHLORIDE | | | | | | | | |
| CETYL ALCOHOL | 2 | 2 | 2 | 2 | 2 | 2 | 2 | 2 |
| CITRIC ACID | 0.6 | 0.6 | 0.6 | 0.6 | 0.6 | 0.6 | 0.6 | 0.6 |

Compositions of Invention examples 1-4 belong to the present invention.
Composition of comparative example 1 does not comprise any divalent metal salt.
Composition of comparative example 2 does not comprise any chelating agent selected from aminocarboxylic acids and salts thereof.
Composition of comparative example 3 does not comprise any divalent metal salt.
Composition of comparative example 4 does not comprise any divalent metal salt and chelating agent selected from aminocarboxylic acids and salts thereof.

### Preparation Process

The compositions were prepared as follows, taking the composition of invention example 1 as an example:

Glycerin and sodium cocoyl glycinate were added into a main beaker, followed by acrylates copolymer with stirring and heating to 70-85° C. Next, magnesium gluconate, trisodium ethylenediamine disuccinate, and potassium chloride were added in order. Next, citric acid and cetyl alcohol were added at a temperature higher than 70° C., then water was added to obtain the composition. The composition was cooled down to 30° C. and aged for 24 hours.

It was found that for the composition of comparative example 2, most of the divalent salt would become crystal together with amino acid surfactant.

### Evaluation of Compositions

The compositions prepared were evaluated on the foam density.

### Foam Density

Foam density was evaluated by observing the foam generated via hands as follows.

Procedure:
1) Wet hands with nitrile gloves and apply 0.5 g of the composition to be tested onto the glove;
2) Rub the composition with two palms for 60 rounds (2 circles/second); and
3) Put together all the foam in one palm.

FIG. 1 shows the foams produced by the composition of invention example 1 in the foam density test.

FIG. 2 shows the foams produced by the composition of comparative example 4 in the foam density test.

A score within a range of 1-15 was given, wherein 1-4 means a low foam density, 5-9 means a middle foam density and 10-15 means a high foam density.

The results obtained were summarized in Table 3.

| Property | IE. 1 | IE. 2 | IE. 3 | IE. 4 | CE. 1 | CE. 2 | CE. 3 | CE. 4 |
|---|---|---|---|---|---|---|---|---|
| Foam density | 12 | 11 | 14 | 13 | 5 | 9 | 6 | 5 |

It can be seen from Table 3, as compared with the composition of comparative examples 1-4, the compositions of invention examples 1-4 show better foam density.

It was also found that the compositions of invention examples 1-4 do not have obvious change in appearance for 2 months at a temperature of 4° C. and 45° C.

1. A composition for cleansing and/or removing makeups from keratin materials, comprising in an aqueous phase:
a) at least one amino acid surfactant;
b) at least one chelating agent selected from aminocarboxylic acids and salts thereof; and
c) at least one divalent metal salt.

2. The composition according to claim 1, wherein the amino acid surfactant is represented by the formula (I): 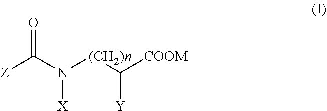
wherein:
Z represents a saturated or unsaturated, linear or branched hydrocarbon group having 8 to 22 carbon atoms,
X is hydrogen or methyl group,
n is0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)OH, —(CH₂)₂C(O)O⁻M⁺, and
M is a salt-forming cation wherein COO is the counter-anion.

3. The composition according to claim 1, wherein
the amino acid surfactant is selected from the group consisting of sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof.

4. The composition according to claim 1, wherein the amino acid surfactant is present in an amount ranging from 1 wt. % to 60 wt. %, relative to the total weight of the composition.

5. The composition according to claim 1, wherein the chelating agent is selected from the group consisting of diethylenetriaminepentaacetic acid (DTPA), ethylenediaminedisuccinic acid (EDDS), trisodium ethylenediamine disuccinate, ethylenediaminetetraacetic acid(EDTA), ethylenediamine-N, N′-diglutaric acid (EDDG), glycinamide-N,N′-disuccinic acid (GADS), 2-hydroxypropylenediamine-N,N′-disuccinic acid (HPDDS), ethylenediamine-N,N′-bis (ortho-hydroxyphenylacetic acid) (EDDHA), N,N′-bis(2-hydroxybenzyl)ethylenediamine-N,N′-diacetic acid (HBED), nitrilotriacetic acid (NTA), methylglycine diacetic acid (MGDA), N-2-hydroxyethyl-N,N-diacetic acid, glyceryl imino diacetic acid, iminodiacetic acid-N-2-hydroxypropyl sulfonic acid, aspartic acid N-carboxymethyl N-2-hydroxypropyl-3-sulfonic acid, beta-alanine-N,N′-diacetic acid, aspartic acid-N,N′-diacetic acid, aspartic acid-N-onoacetic acid, chelating agents based on iminodisuccinic acid (IDSA), ethanoldiglycine acid, and tetrasodium glutamate diacetate (GLDA).

6. The composition according to claim 1, wherein the chelating agent is present in a content ranging from 0.05 wt. % to 5 wt. %, relative to the total weight of the composition.

7. The composition according to claim 1, wherein the divalent metal salt comprises a metal ion M1²⁺ selected from the group consisting of Mg²⁺, Ca²⁺, Zn²⁺, Fe²⁺, Mn²⁺, and Cu²⁺.

8. The composition according to claim 1, wherein the divalent metal salt is present in an amount ranging from 0.1 wt. % to 5 wt. %, relative to the total weight of the composition.

9. The composition according to claim 1, further comprising at least one C₂-C₈ polyol selected from the group consisting of butylene glycol, propylene glycol, dipropylene glycol, isoprene glycol, hexylene glycol, caprylyl glycol, glycerin and polyethylene glycols.

10. The composition according to claim 9, wherein the C₂-C₈ polyol is present in an amount ranging from 1 wt. % to 70 wt. %, relative to the total weight of the composition.

11. The composition according to claim 1, wherein the aqueous phase represents from 40 wt. % to 70 wt. %, relative to the total weight of the composition.

12. The composition according to claim 1, comprising in an aqueous phase, relative to the total weight of the composition:
a) from 35 wt. % to 45 wt. % of at least one amino acid surfactant selected from the group consisting of sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, and a mixture thereof;
b) from 0.2 wt. % to 0.4 wt. % of at least one chelating agent selected from the group consisting of ethylenediamine-tetraacetic acid (EDTA), diethylenetriaminepentaacetic acid (DTPA), S,S′-ethylenediaminedisuccinic acid(EDDS), trisodium ethylenediamine disuccinate, ethylenediaminetetramethylenephosphonic acid (EDTMP), and tetrasodium glutamate diacetate (GLDA), and mixtures thereof, and
c) from 0.5 wt. % to 1 wt. % of at least one divalent metal salt selected from the group consisting of magnesium gluconate, magnesium PCA, magnesium sulfate, magnesium acetate, magnesium carbonate, magnesium aluminum silicate, calcium carbonate, calcium PCA, calcium chloride, calcium carbonate, zinc PCA, zinc gluconate, copper PCA, copper gluconate, and a mixture thereof.

13. A non-therapeutic process for cleansing and/or removing makeups from keratin materials, comprising the application to the keratin materials of the composition according to claim 1, and rinsing off said composition after a period of time.

14. The composition according to claim 2, wherein M is selected from the group consisting of sodium, potassium, ammonium, and triethanolamine.

15. The composition according to claim 2, wherein
Z represents a linear or branched C₈-C₂₂ alkyl group,
X is a hydrogen or methyl group,
n is0,
Y is selected from the group consisting of hydrogen, —(CH₂)₂C(O)OH, and —(CH₂)₂C(O)O⁻M⁺, and
M is a counter-anion, selected from the group consisting of sodium, potassium, ammonium, and triethanolamine.
